(19) United States
(12) Patent Application Publication
(10) Pub. No.: US 2023/0073613 A1
(43) Pub. Date: Mar. 9, 2023

(54) AUTOMATIC CLASSIFICATION OF HEART SOUNDS ON AN EMBEDDED DIAGNOSTIC DEVICE

(71) Applicants: Jeffery Alan Lettman, Petersburg, FL (US); Joshua Valete Hopkins, Petersburg, FL (US)

(72) Inventors: Jeffery Alan Lettman, Petersburg, FL (US); Joshua Valete Hopkins, Petersburg, FL (US)

(21) Appl. No.: 17/702,724

(22) Filed: Mar. 23, 2022

Related U.S. Application Data
(60) Provisional application No. 63/165,018, filed on Mar. 23, 2021.

(51) Int. Cl. A61B 5/00; H04R 1/46; A61B 7/04; A61B 7/00; A61B 5/024; A61B 5/318

(52) U.S. Cl. CPC A61B 5/7264; H04R 1/46; A61B 7/04; A61B 7/003; A61B 5/02444; A61B 5/318; A61B 5/7221

(57) ABSTRACT

An automatic diagnostic apparatus and corresponding method is disclosed for recognizing heart sounds of interest, i.e., murmurs, detected in streaming audio data picked up by a stethoscope. Sensors included in the device capture audio data in real time during an auscultation exam performed by a physician. A feature vector that models the stream of audio data is created and supplied to a deep neural network stored on the diagnostic device. The deep neural network generates a probability for each of the heart sounds of interest. When the probability of detection exceeds a pre-established threshold value the device alerts the physician through visual and/or audio cues, enhancing the physician's diagnostic capability during routine examination.

### CROSS-REFERENCE TO RELATED APPLICATIONS

The present application claims priority to U.S. Provisional Application No. 63/165,018 filed Mar. 23, 2021, titled “AUTOMATIC CLASSIFICATION OF HEART SOUNDS ON AN EMBEDDED DIAGNOSTIC DEVICE,” which is hereby incorporated by reference in its entirety.

### TECHNICAL FIELD

Embodiments of the invention relate generally to cardiac auscultation, particularly in the case of systems and methods for automatically classifying detected heart sounds on an embedded diagnostic device.

### BACKGROUND

Cardiac auscultation is performed in virtually every clinical examination and can identify heart disease before the symptoms are present and apparent to the patient. However, in practice, the majority of heart murmurs are missed, due to their subtlety or other conditions during the examination. Even significant valvular heart disease is identified by auscultation in less than 43% of actual cases.

Devices thus far require a connection to a mobile app and/or the cloud to complete the intensive processing involved in running a classification algorithm. UI/UX and visual feedback in these cases is provided on the mobile app, after processing is completed. These devices generally require connection to a mobile app or computer, a high-speed internet connection, and the use of a smartphone. This significantly limits the conditions under which they can be used. Additionally, these devices typically operate at a delay, record data, and transmit this data to another device or devices for analysis prior to providing a result.

The present invention's electronic device can be attached to a standard or regular stethoscope and can be used to automatically flag or provide an indication to the physician or medical personnel that heart murmurs have been detected in the patient in real-time, during routine examinations. No other devices provide heart murmur detection in real-time, on a standalone device. This device performs the processing required to run one or more detection algorithms on-board in real-time. For this reason, the device does not require a connection to external computing resources. The device can signal a user directly once a prediction or analysis is reached. This allows the device's use to be more readily integrated into standard auscultation exams, improves ease of use, and efficiency.

Thus, the need exists for real-time detection of internal medical issues using embedded electronic devices which can be coupled with standard stethoscopes and/or other medical equipment.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

The example embodiments provided herein relate to and disclose techniques for artificial intelligence detection of abnormal heart sounds classified as “heart murmurs” (HM). The detection of HMs in the medical field is difficult and leveraging technology to assist in detecting said HMs in the patient population when physicians listen to a patient's heart sounds in real time (also referred to as “auscultation” for this application).

In some embodiments described herein, systems, methods, and devices that automatically detect heart murmurs during auscultation includes an apparatus attached to a stethoscope during use, circuitry configured to determine if a heart murmur is present, and a sensory method such as lights or audio feedback to communicate to the user if a heart murmur is present.

In some embodiments described herein, systems, methods and devices for automatic detection of an internal body signal of interest in a stream of diagnostic data using a trained classifier deployed on an embedded electronic device. These systems, methods, and devices can include classification data that classifies the internal body signal, or heart sound as an internal body signal of interest, or normal heart sound or a certain type of heart murmur, based on a preestablished training set of selected types of heart murmurs, and analyzing the heart sounds in real time by sending into an input layer of a neural network. The neural network and associated weights are trained on this preestablished training set of select types of heart murmurs prior to implementation in the analyzation method during real time analysis during auscultation.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a block diagram of an embedded electronic device for use in the automatic detection of internal body sounds of interest, according to some embodiments;

FIG. 2 illustrates a diagram of an embedded electronic device for cardiac auscultation, according to some embodiments;

FIG. 3A-3B illustrate diagrams of an embedded electronic device for use in the automatic detection of internal body sounds of interest, including a first operating condition and a second operating condition, according to some embodiments;

FIGS. 4A-4B illustrate a device as installed on a stethoscope, according to some embodiments;

FIG. 5 illustrates a flowchart diagram of an embedded electronic device data flow, according to some embodiments;

FIG. 6 illustrates a flowchart diagram of an embedded electronic device use case, according to some embodiments;

FIG. 7 illustrates a flowchart diagram of a supervised neural network training data flow, according to some embodiments;

FIG. 8 shows an example embodiment diagram including a cross-section view of an embedded electronic device;

FIG. 9 shows an example embodiment diagram including a cross-section view of an embedded electronic device; and

FIG. 10 shows an example embodiment diagram including a cross-section view from the top of an embedded electronic device.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The present embodiments include embedded electronic devices designed to connect or couple in-line to a stethoscope so that it can record audio signals being transmitted in the airway of the stethoscope tubing. An example device can comprise: one or more processors; non-transitory computer readable memory, which stores a compressed deep neural network and instructions for the microprocessor; at least one audio receiver or transceiver, such as a microphone, operable to receive and/or record the sound transmitted through stethoscope tubing; a means of sensory output to communicate the results to the physician; and an operable coupled battery to power the device.

The present embodiments also include systems, methods, and devices for automatic detection of internal body signal of interest (e.g. a heart sound, lung sound, digestive sound, electrical sound, or other sound), in a stream of audio data, recorded by the device when connected to a stethoscope, using a trained classifier deployed on an embedded electronic device. An example embodiment method can comprise:

A) Training by an audio recognition system that includes at least one computer, a deep neural network to determine probabilities that data received by the deep neural network has features similar to key features of heart sounds of interest, the training comprising: providing the deep neural network with a first set of feature values for heart sound data; adjusting values for each of a plurality of weights included in the neural network; and compressing the plurality of weights and optimizing based on a balance of performance and size for deployment onto resource-constrained embedded systems.

B) Deploying the deep neural network, which was previously trained and compressed, on an embedded electronic device.

C) Acquiring on the embedded electronic device, streaming data detected by a sensor (e.g. audio from a microphone on the device, electrical signals from an EKG sensor, or others), and providing said data stream to the on-device deep neural network.

D) Using the trained deep neural network to determine a probability that data received by the deep neural network has features similar to key features of a body signal of interest (e.g. one or more heart sounds, lung sounds, digestive sounds, electrical signals, or others), the deep neural network to detect only those of the one or more body signals of interest encoded in a stream of audio data. These features can be time-frame data, frequency, amplitude, irregularities, or others.

E) Sending a notification of the detection of an internal body signal of interest to an output device when the probability of detection exceeds a pre-established threshold value or another trigger occurs.

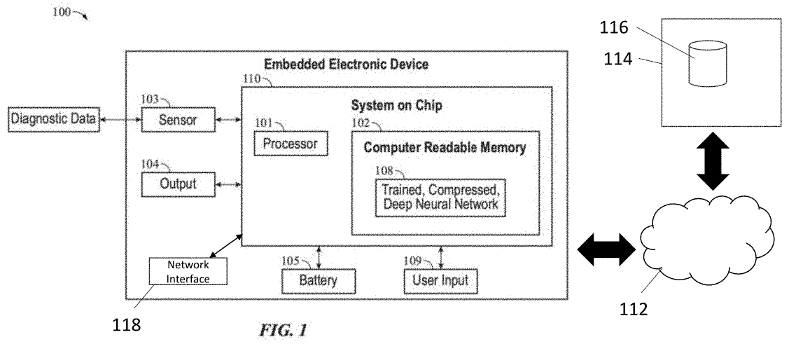

FIG. 1 illustrates a block diagram 100 of an embedded electronic device for use in the automatic detection of internal body sounds of interest, according to some embodiments. As shown, an embedded electronic device can include a system on a chip 110. This system on a chip 110 can include a processor 101 (also referred to herein as a microprocessor) a non-transitory computer readable memory 102, and a trained, compressed deep neural network 108 stored in the memory 102. Also included in the embedded electronic device (or coupled thereto, in some embodiments) can be at least one sensor 103, an output 104, battery 105, user input 109, and others, as appropriate.

In an example embodiment, sensor 103, which can be a microphone or other transducer or signal detecting/receiving component(s) in various embodiments, detects, records and/or senses sounds in, from, or through a stethoscope or phonendoscope. These sounds are then transduced into electrical signals and provided as data to communicatively coupled processor 101. Processor 101 can in turn run one or more processes that are stored in non-transitory memory 102 that may compare the electrical signals against, with, or through the deep neural network 108 to determine whether they match or indicate a particular type of internal body process or condition of the individual being monitored. In some embodiments thresholds can be used, as well as markers, quantities of matching indicators, or others to determine whether the signal matches known body processes.

Conditions can be classified in some instances. For example, a heart sound may be classifiable as one or more types of heart murmurs, which can be associated with a particular diagnosis. More generally heart murmurs can be considered abnormal heart sounds. Classified murmurs may further be classifiable by their intensity into different grades.

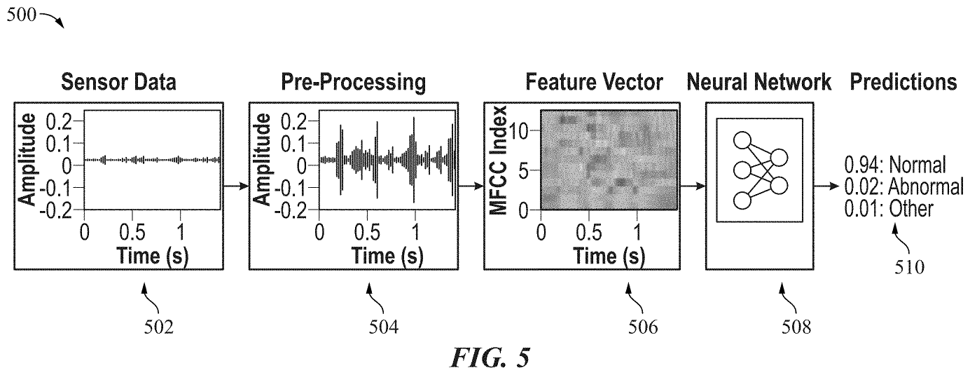

To elaborate, the computer readable memory 102 stores the trained, compressed neural network 108, and the instructions for running the microprocessor 101. The processor 101 prepares the streaming audio data for the trained, compressed deep neural network 108 by performing feature extraction and creating a feature vector which creates one or more virtual models of the audio data. The feature vector can be provided to the deep neural network 108, which in turn provides a prediction regarding the occurrence of the heart sounds of interest in the streaming data. See FIG. 5 and associated description for further information. When this prediction exceeds a specified threshold, the processor 101 can alert a physician, nurse, technician, or other user who is using the device through output 104, by causing output 104 to emit an indication. In various embodiments, output 104 can include one or more visual indicators such as light emitting diode (LED) light flashes, pulses, or constant lighting; audible indicators such as sounds emitted from an audio emitting speaker or transceiver; physical indicators, such as vibration or other physical sense indicators from a motor or component having movement; combinations of these; and/or others. Battery 105 can be directly or indirectly coupled to system on a chip 110, output 104, and/or other components as necessary for functionality described herein.

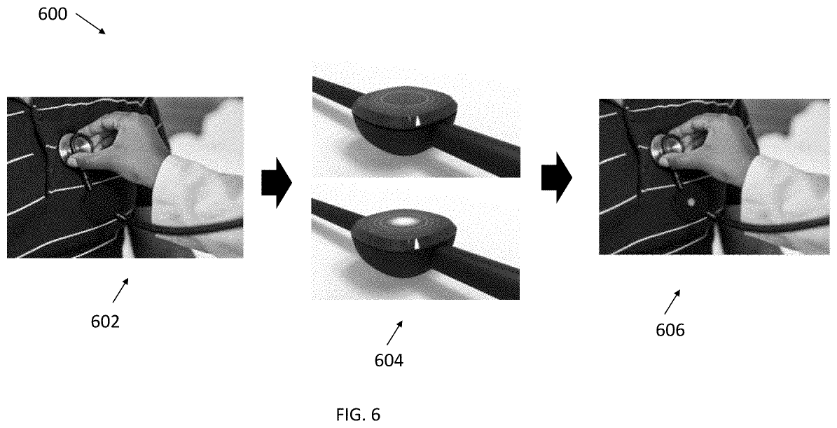

A user input 109 can include one or more buttons or screens in various embodiments that allow a user to interact with the device. Functions can include power on/off, standby, activate, deactivate, acknowledge signal, change mode, reset, or others, in various embodiments. See FIG. 6 and associated description for a use case explanation.

In various embodiments, the device may not require any outside data or direct power connection to function. Battery 105 can be charged prior to dissemination to a physician and may or may not be rechargeable through wireless or wired connection in various embodiments. In some embodiments, one or more network interface(s) can be included such that the embedded electronic device can receive software updates and to extract or send stored information via a network connection to a receiving device such as a computer, mobile device, server, or other device.

The embedded electronic device is able to perform its classification activities/processes on received data signals using the pre-trained (used interchangeable with trained, herein) deep neural network (DNN) 108. In many embodiments, the DNN 108 has been compressed (quantized) for running on resource constrained platforms. Incoming monitored or sensed audio data from sensor 103 is checked using the deep neural network 108 can also be compressed in various embodiments. In various embodiments, the data stream can be compressed to a standard format (e.g. .wav, .mp3, .mp4, or many others, known or later developed) after being processed to save for later review on a separate device (e.g. a computer, smartphone, tablet computer, or other computing device). The DNN 108 can be designed and optimized specifically for this resource constrained application on the embedded electronic device in some embodiments. Moving the DNN 108 from the cloud, where it would typically be stored, and directly onto the device can be important to functionality of this device, as it allows the feedback, in the form of output 104, to be provided in real-time or near real-time to the physician or other user, and it eliminates the standard use requirement of a peripheral devices, such as a smartphones.

The deep neural network 108 needs to be trained specifically for this resource constrained application and compressed for deployment onto the device. The DNN is trained using a database of heart sounds prior to deployment on the device. The DNN can be used to determine probabilities that data received by the DNN has features similar to key features of heart or other internal body sounds of interest. Training of the DNN can be performed by at least one computer, following the steps of providing the DNN with a first set of feature values for heart (or other) sound data, adjusting values for one or more weights included in the neural network, and compressing the plurality of weights and optimizing based on balance of performance and size for deployment onto resource constrained embedded systems.

There are two approaches to training, supervised and unsupervised. In some embodiments, supervised training can be used for embedded electronic device DNN training. In other embodiments, unsupervised training can be used for embedded electronic device DNN training.

In supervised training embodiments, both inputs and the outputs can be provided. The DNN then processes the inputs and compares resulting outputs against the desired outputs. Errors can then be propagated back through the system, causing the system to adjust the weights which control the network. This process can iteratively occur numerous times over and over as the weights are continually tweaked. The set of data which enables the training is called a “training set.” During the training of a network the same set of data is processed many times as the connection weights can be ever refined.

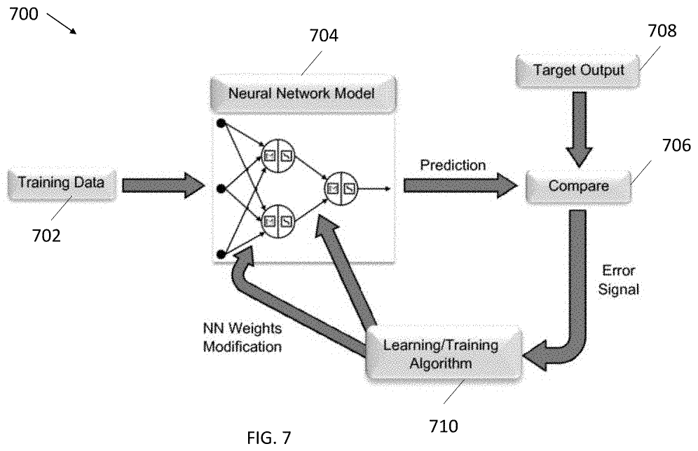

In unsupervised training, the DNN can be provided with inputs but not with desired outputs. The DNN itself can then decide what features it will use to group the input data. This is often referred to as self-organization or adaption. See FIG. 7 and associated description for additional information about DNN training.

Critically in our application we implemented learning under resource constraints. This departs from traditional machine learning in that model features are accompanied by costs (e.g. memory required, processing time, etc.). This is what allows us to deploy our trained model on small, embedded platforms.

In some embodiments, a network interface 118 can be provided that allows the embedded device to receive and/or send data via network 112 to and/or from one or more devices 114. Devices can include smartphones, tablets, desktop and/or laptop computers, servers, proprietary computing devices, wearable devices such as smartwatches and smart glasses and others in various embodiments. In some embodiments, one or more databases 116 can be stored in non-transitory memory on device 114. Memory and/or databases 116 of device 114 can also store DNN and other information such as patient information, measurements, medical data, algorithms, and others, as appropriate and necessary.

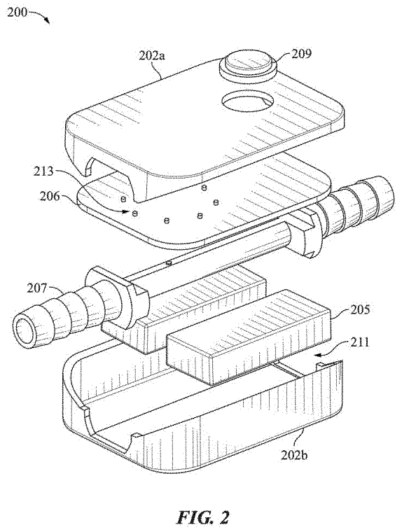

FIG. 2 illustrates a diagram 200 of an embedded electronic device for cardiac auscultation, according to some embodiments As shown, in various embodiments many of the electronic components described herein are assembled on a printed circuit board assembly (PCBA) 206 or multiple coupled PCBAs. In some embodiments, a PCBA 206 can include some or all of the components shown in the diagram of FIG. 1. In some embodiments, a microphone of PCBA 206 needs or requires access to stethoscope tubing in a manner that maintains an airtight seal, so false signals are not captured and/or real signals are not interfered with. This can be accomplished through the inclusion of custom tubing 207, which can be in-line between an existing stethoscope chest piece and stethoscope binaural assembly. In other embodiments, access and/or an airtight seal can be provided by puncturing existing stethoscope tubing and to gain access to its interior and coupling the microphone portion in appropriate fashion. The microphone may be attached, joined to, coupled with, or otherwise connected with tubing 207.

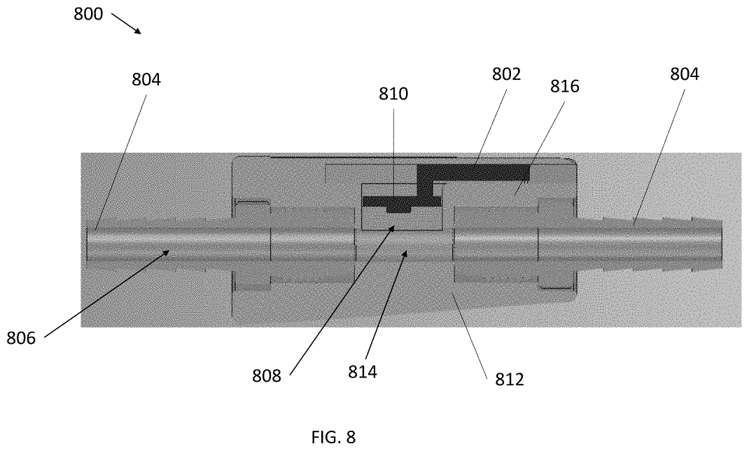

In some embodiments, stethoscope tubing can or may be punctured, whereafter positioning a microphone in, at, or otherwise adjacent or near the opening can allow it to adequately detect audio signals in the tubing. Potting with silicone or similar sealant can be used to create an airtight seal between the punctured tube and the housing around or near the microphone(s). See FIGS. 8-10 and associated description for additional detail.

In some embodiments, an airtight seal may not be required for adequate signal detection by a microphone (e.g. for audio signals) or other sensor (e.g. electrical signals for EKG detection). In such embodiments no puncture, hole, or other access to the interior of a stethoscope tube may be required. Clasp(s), clamp(s), and/or other coupling mechanisms can be used in such embodiments. In some embodiments, more than one type of internal signals can be monitored and analyzed by the embedded electronic device (e.g. heart signals, electrical signals, lung signals, and/or others).

In some embodiments digital stethoscopes can be used with embedded electronic devices. In such embodiments, the digital stethoscope may capture data on its own, which can be communicatively coupled with an embedded electronic device in order to employ DNN(s) to achieve the outcomes outlined herein.

One or more housing, which can be plastic in some embodiments, can include an upper housing 202a and a lower housing 202b. These upper and lower housings 202a, 202b can be permanently coupled together in some embodiments or removably coupled using any manner of detents, buttons, latches, seals, glues, resins, epoxies, screws and receiving holes, or other appropriate coupling mechanisms. Upper and lower housings 202a, 202b can be contain the PCBA(s) 206 and tubing section 207. When coupled, housings 202a, 202b can provide at least one hole 211 that tubing section 207 passes through, but which is flush with the exterior surface of tubing section 207. As such, the components on the interior of the housing can be protected from moisture, dirt, dust, or other corrosive or damaging elements.

As shown, at least one battery 205 can be included and housed within housing 202a, 202b of an assembled device. Battery 205 can be charged through induction in some embodiments, while in other embodiments a plug or hole can be provided to allow for removably coupling a wire to charge the battery, as is known in the art. Battery 205 can be coupled directly to PCBA 206 to provide power.

An indicator 213 can include one or more visual, audio, mechanical, or other mechanisms to alert a user and/or indicate to a user a particular operating status or state of the device is currently in (e.g. on, activated (processing data), condition identified, incorrect use (e.g. not at appropriate site, moved during use, or others), unknown condition (please retry), low power, charging, prediction confidence level, software updating, audio recording, standby, monitoring, resetting device, paired with other device (e.g. via Bluetooth or other wireless connection), device error state, second body signal detected (e.g. heart murmur, lung sound, arrhythmia, or others) or others). As such, the output provided to the user could take different forms, lights, audio, tactile feedback. In the example embodiment, PCBA 206 can have one or a plurality of LED indicator light(s) 213 included, that are able to shine through holes, a membrane, or clear or opaque section or surface of upper housing 202a to indicate a condition to the user. The resulting output can also be communicated to a separate device which provides an indication in some embodiments (e.g. wirelessly transmitted signal to a related and communicatively coupled device such as a speaker, mechanical indicator, and/or audible indicator.

One or more user input mechanisms 209 can be included in various embodiments. As shown, input mechanism 209 can be a button can be a separate from an upper housing 202a, or could be integrated in some embodiments. When actuated or engaged, the button can cause the processor of the PCBA 206 to perform and/or cease a function. Input mechanism 209 could also be a touchscreen display or other mechanism as appropriate to allow a user to interact with and control the device.

In various embodiments the device is able to recognize a number of sounds and/or types of sounds and is not limited to heart sounds. These can include lung sounds, digestive tract sounds, or others, as appropriate.

In various embodiments, diagnostic data being provided to the device is not limited to that which could be picked up with a microphone. As such, electrical signals produced by internal organs, such as those picked up by electrocardiograms, can be detected.

In an example embodiment, one or more usage steps after assembly can include: 1. Physician positions the stethoscope diaphragm at a first auscultation site. 2. Physician presses an activation button 209 on the device to start processing. 3. Physician listens to the heart sounds while the device processes data. This step is expected to take a particular amount of time (e.g. about milliseconds, fractions of a second, multiple seconds, five seconds, or other orders of magnitude may be employed in various embodiments) or range of time, during which the physician keeps the diaphragm pressed to the auscultation site. 4. The device signals (e.g. visually by flashing or shining an LED light) one of three possible results. a. Heart Murmur detected. b. Heart murmur not detected. or c. Unknown results, please try again. 5. Physician can then move the stethoscope diaphragm to a next auscultation site and repeats steps 1-4, if additional measurements are desired.

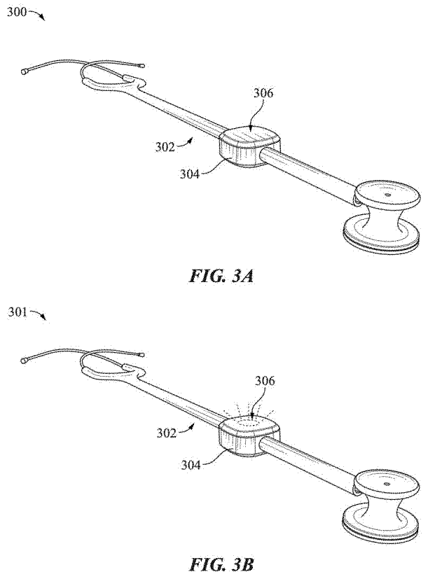

FIG. 3A-3B illustrate diagrams 300, 301 of an embedded electronic device for use in the automatic detection of internal body sounds of interest, including a first operating condition and a second operating condition, according to some embodiments. As shown an embedded electronic device 304 can be operably coupled with a stethoscope 302 at some point along the length of the tube or hose of the stethoscope 302. An indicator 306 can be off when not indicating anything, as in diagram 300, or on when indicating something, as in diagram 301.

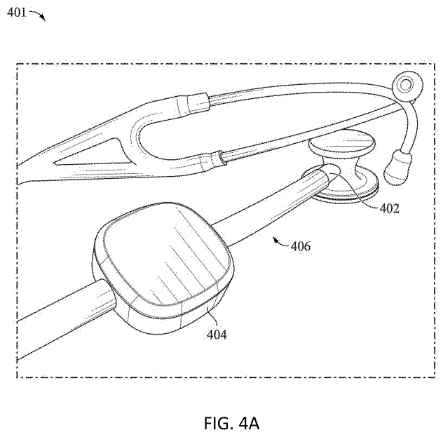

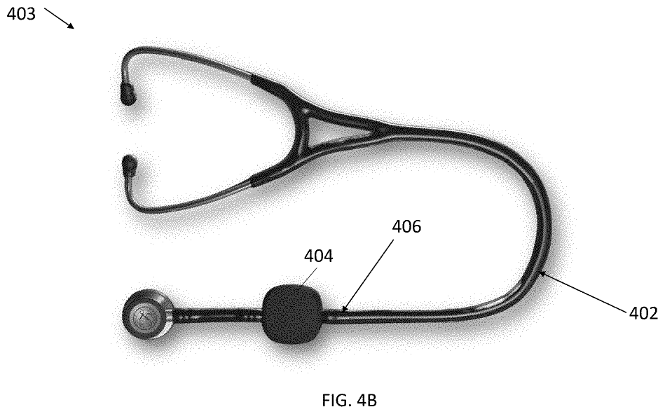

FIGS. 4A-4B illustrate diagrams 401, 403 of an embedded electronic device 404 as installed on a stethoscope 402 from different viewpoints, according to some embodiments. As shown, tubing 406 of the embedded electronic device 404 can be coupled over and around tubing of the stethoscope 402 in some embodiments. The shape of embedded electronic device 404 and its surfaces and faces can be generally squarish or cube-like in some embodiments, oval, circular, cylindrical, spherical, or others in various embodiments, in some embodiments, indicators may protrude, be flush with, or embedded within the device, so long as they serve their stated indicating purpose.

In some embodiments, additional features can be included. In some embodiments, one or more additional microphones can be included that are outward facing and signals captured thereby be used by the processor to perform noise-cancelling operations on the stethoscope audio recording input data stream to provide more accurate overall results. In some embodiments, a multi-tiered neural network approach can be implemented. In such instances, a first deep neural network can identify a snippet of captured data of interest and a second (or multi-leveled operating) deep neural network can function as a classifier or other mechanism.

In a multi-tiered deep neural network a first network can be used to segment streaming audio, so as to identify or pull a relevant snippet of the streaming data out so that it can be run through or otherwise used by a second neural network to obtain prediction(s) about conditions which may indicated by signals or data present in that snippet.

To elaborate, in some embodiments: An audio heart sound can include of a first heart sound, S1, followed by a second heart sound, S2. A time existing between these sounds, and in between successive groupings of sounds can be related to heart rate and may vary by patient. A first stage of the deep neural network can be used to recognize S1 and S2 sounds, so as to decompose or otherwise break down the streaming audio signal into meaningful cardiac events. These identified events can then be provided to a second stage of the deep neural network for recognition of conditions (i.e. heart murmurs or other conditions). Providing cleanly isolated cardiac events can improve the accuracy of the system in some instances.

FIG. 5 illustrates a flowchart diagram 500 of an embedded electronic device data flow, according to some embodiments. As shown, a sensor data step 502 can include raw data collection from a sensor of an embedded electronic device, which can be in-line in various embodiments (e.g. audio data captured using a microphone coupled with a stethoscope tube). Next, a pre-processing step 504 can include filtering, amplifying, active noise canceling, or other operations on the data. A feature vector step 506 can include feature extraction. Features can be time-domain data, frequency-domain data, spectral data, or others as appropriate. Next, a neural network step 508 can include employment and/or use of a DNN, which may include two or more layers, to identify potential matches. This can include one or more convolutional layers in some embodiments. Finally, a prediction step 510 can include determination of the likelihood that the data matches or indicates existence of a particular condition. Various thresholds and/or ranges can be used in different embodiments. As shown, a 0.94 output could indicate normal behavior, 0.02 could indicate an abnormal behavior, and 0.01 could indicate another issue (e.g. equipment malfunction, failure to capture data sufficiently, or others).

FIG. 6 illustrates a flowchart diagram 600 of an embedded electronic device use case, according to some embodiments. As shown in the example embodiment, a first step 602 can be a user, such as a medical professional (e.g. doctor or nurse) or other technician, positioning the chest piece of a stethoscope with coupled embedded electronic device at a site on the patient for detection. A next step 604 can include the medical professional activating the embedded electronic device (e.g. by pressing a button of the device or otherwise engaging with a user input interface of the device). The device may then indicate (e.g. by one or more of an audible sound from a speaker, lighting of indicator light(s), changing color of indicator light(s), blinking or flashing indicator light(s), or others) to the user, whereafter the user can listen for sounds or have the device check for other signals (in the case of an EKG). In a next step 606, the device can indicate with a particular color, sound, flashing, or other mechanism that a condition has been detected, no condition has been detected, or there was a problem. As an example, a red light may indicate an abnormal heart murmur has been detected. A green light may indicate normal heart sounds were detected. A yellow light may indicate an undetermined or indeterminate result. This indication may take a particular amount of time before being displayed, played, or otherwise indicated. For example, five seconds may pass, 10 seconds, 15 seconds, or another amount of time or range of time. It should be understood that in various embodiments different time amounts can be employed and faster results may be indicated if desired (with reduced accuracy) or slower results may be indicated (with increased accuracy in many instances).

FIG. 7 illustrates a flowchart diagram 700 of a supervised neural network training data flow, according to some embodiments. As shown, training data 702 can be provided to a neural network model 704, which can then output a prediction. The prediction can be compared in step 706 with a target output 708, which can indicate an error signal. The error signal can then be used by a learning and/or training algorithm in step 710, which can output neural network weight(s) modifications that are implemented by the neural network model 704 in further iterations. Further information about neural network training is provided at (https://www.researchgate.net/publication/299390844 The Development of Neural Network Based System Identification and Adaptive Flight Control for an Autonomous Helicopter System).

FIG. 8 shows an example embodiment diagram 800 including a cross-section view of an embedded electronic device. As shown in the example embodiment, a PCBA 802 including a microphone can be positioned in a space between and abutting barbed connectors 804 of a tubing 806. PCBA 802 can generally be adjacent to a central portion of tubing 806. An area above the microphone PCBA 802 can be potted with silicone to create a seal in some embodiments. Stethoscope tubing leading to a chest piece can be connected to or otherwise coupled with barbs 804 extending from one side of the embedded electronic device, for example to the right, and stethoscope tubing connected to and extending to earpieces of the stethoscope can be connected to or otherwise coupled with the barbs 804 extending from an opposite side of the embedded electronic device, for example to the left. Other configurations are possible and may be desirable in other embodiments (e.g. both coming from one side, one extending out the front such that they are perpendicular, or others).

In some embodiments, a chamber 808 between the exit side of two barbed connectors 804 is provided. The top of the chamber 808 can including an opening for a microphone 810 of or coupled with PCBA 802.

A rigid structure 816 within housing 812 designed with an internal tube structure 814. This tube structure or chamber 814 can be integrated in a monolithic structure with or otherwise be coupled to barbed connectors 804 on either end to connect to the stethoscope tubing. The PCBA 802 can be mounted within housing 812 to the top of this rigid structure 816, and the structure 816 can have an opening or access to the interior or into the tube section 814 which aligns with the position of the microphone 810 on the PCBA 802. Securing the PCBA 802 to the top of this rigid structure can create a seal, for example an airtight seal, in some embodiments.

In some embodiments at least one noise cancelling microphone can be included that has access to and can receive external sound signals that can be processed and used to improve the accuracy of results and predictions by countering and/or removing background noise from an audio sample.

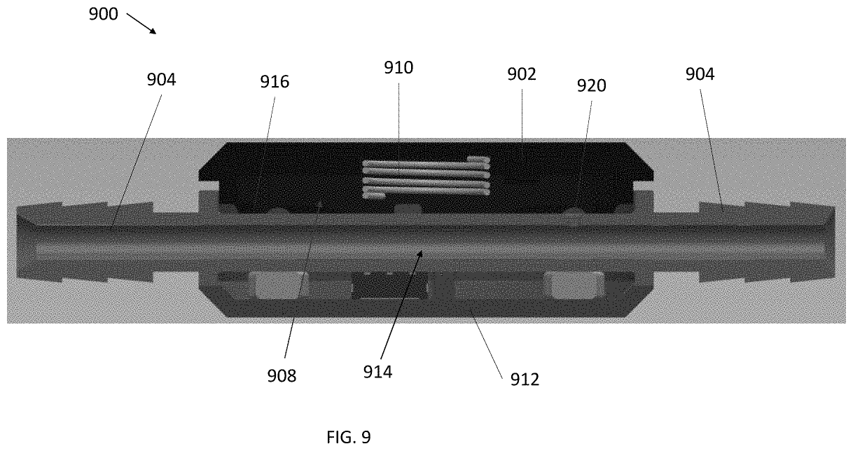

FIG. 9 shows an example embodiment diagram 900 including a cross-section view of an embedded electronic device. As shown in the example embodiment, an upper surface of rigid structure 916 can be a location where the PCBA 902 is coupled or attached. A hole 920 in the main tube 914 which can be access for a microphone port. The microphone 910 on the PCBA 902 is bottom ported, so the PCBA 902 creates the seal against the tube. The stethoscope tubing leading to the chest piece connected to the barbs 904 on one side, and the stethoscope tubing connected to the earpieces was connected to the barbs 904 on other side.

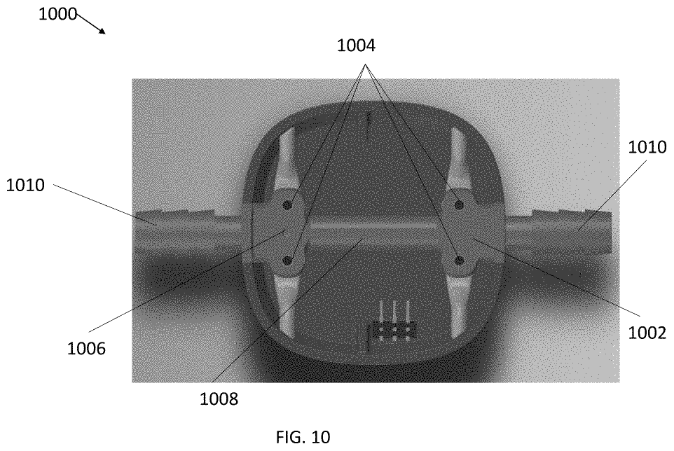

FIG. 10 shows an example embodiment diagram 1000 including a cross-section view from the top of an embedded electronic device. As shown in the example embodiment, with a PCBA and top piece removed for visibility of other structures. A surface 1002 can be a location where a PCBA is attached or coupled, for example using screws, glues, or other securing mechanisms and/or media. One or more holes 1004 (e.g. four holes in the example embodiment) on, at, or through this surface can be threaded mounting holes for receiving screws that can secure the PCBA. A port hole 1006 can extend through a wall of main tube 1008 which allows access for a microphone and/or other sensor, which can have a microphone or other port in some embodiments. The microphone on the PCBA can be bottom ported, so the PCBA can create the seal against the tube in some embodiments. Stethoscope tubing leading to the chest piece connected to or coupled with barbs 1010 on one side of the embedded electronic device, and stethoscope tubing connected to earpieces can be connected to or coupled with barbs 1010 on the other side in some embodiments.

In some embodiments, combinations of multiple sensors (e.g. multiple microphones, one microphone and one EKG monitoring sensor, or others) can be included in a single electronic embedded device. These may be in line or at strategic locations and can be used to increase the accuracy of results.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. An embedded electronic device, comprising:
at least one sensor configured to sense a stream of diagnostic patient data;
non-transitory computer readable memory;
at least one processor;
a deep neural network stored in the non-transitory computer readable memory;
at least one indicator; and
a battery,
wherein the stream of diagnostic data is inputted into the deep neural network for analysis and the indicator indicates to a user of the device that an internal body signal of interest has been detected in the stream of data based on the results of the deep neural network analysis.

2. The electronic device of claim 1, wherein the diagnostic patient data is acoustic data picked up by a stethoscope.

3. The electronic device of claim 2, wherein the internal body signals of interest are heart sounds, which can include heart rate.

4. The electronic device of claim 2, wherein the internal body signals of interest are lung sounds.

5. The electronic device of claim 2, wherein the internal body signals of interest are electrical signals produced by internal organs, and are sensed by an electrocardiogram (EKG) sensor of the device.

6. The electronic device according to claim 3, wherein the heart sounds are classifiable as normal or abnormal.

7. The electronic device according to claim 3, wherein the hear sounds are further classifiable as types of murmurs.

8. The electronic device of claim 6, wherein the types of murmurs are further classifiable by grade.

9. The electronic device of claim 2, wherein the device connects in-line with a stethoscope chest piece, binaural, or earpiece.

10. The electronic device of claim 2, wherein the stethoscope is a digital stethoscope, and the device can be configured to receive a digitized signal from the digital stethoscope.

11. The electronic device according to claim 1, wherein the sensor is receiving a stream of audio data through a microphone.

12. The electronic device of claim 1, wherein the operations additionally include:
generating a confidence score by combining two or more consecutive probabilities for the same internal body signal of interest, the consecutive probabilities corresponding with feature vectors that model different consecutive portions of the stream of data from an internal body provided by the one or more sensors; and
determining whether said stream of data includes the internal body signals of interest using the generated confidence score.

13. The electronic device of claim 1, wherein the sensory output can be provided via a separate device connected physically or wirelessly to the embedded electronic device.

14. A method of automatic detection of an internal body signal of interest in a stream of diagnostic data using a trained classifier deployed on an embedded electronic device, the method comprising:
a processor using data from a trained deep neural network stored in non-transitory computer readable memory to determine a probability that diagnostic data received by the deep neural network has features similar to key features of at least one internal body signals of interest; and
the processor causing an indicator to indicate detection of an internal body signal of interest based on the deep neural network outputting a value that exceeds a pre-established threshold value.

15. The method of claim 14, wherein the diagnostic data is acoustic data picked up by a stethoscope.

16. The method of claim 15, wherein the internal body signals of interest are heart sounds.

17. The method of claim 15, wherein the internal body signals of interest are lung sounds.

18. The method of claim 16, wherein the heart sounds are classifiable as normal or abnormal.

19. The method of claim 18, wherein the heart sounds are further classifiable as types of murmurs.

20. The method of claim 19, wherein the types of murmurs are further classifiable by grade.
